(19) United States
(12) Patent Application Publication
(10) Pub. No.: US 2022/0260573 A1
(43) Pub. Date: Aug. 18, 2022

(54) METHOD OF DISCRIMINATING LUNG CANCER PATIENTS

(71) Applicant: BIOMARK CANCER SYSTEMS INC., Richmond, BC (CA)

(72) Inventor: Rashid Bux, Richmond, BC (CA)

(21) Appl. No.: 17/630,989

(22) PCT Filed: Jul. 29, 2020
(86) PCT No.: PCT/CA2020/051041

§ 371 Date: Jan. 28, 2022

Related U.S. Application Data
(60) Provisional application No. 62/880,062, filed on Jul. 29, 2019.

(51) Int. Cl. G01N 33/574

(52) U.S. Cl. CPC G01N 33/57423; G01N 2405/04

(57) ABSTRACT

A biomarker panel for discriminating lung cancer comprises the biomarkers valine, lysoPhosphatidylcholine acyl C18:2 (Lyso PC a18:2), decadienyl-L-carnitine (C10:2) phosphatidylcholine, acyl-alkyl C36:0 (PC aa C36:0), phosphatidylcholine diacyl C30:2 (PC aa C30:2), spermine, and diacetylspermine.

### CROSS-REFERENCE TO RELATED APPLICATION

This application claims the benefit of, and priority to, U.S. provisional patent application No. 62/880,062 filed Jul. 29, 2019, the entire contents of which are incorporated by reference herein.

### BACKGROUND

### Field of the Disclosure

The present disclosure relates to a method of diagnosing cancer and, in particular, to a method of discriminating lung cancer patients as a diagnostic and treatment monitoring tool.

### Description of the Related Art

Lung cancer is the most common cause of cancer-related deaths worldwide. Early diagnosis is crucial to increase the curability chance of the patients. Low dose CT screening can reduce lung cancer mortality, but it is associated with several limitations. Metabolomics is a promising technique for cancer diagnosis due to its ability to provide chemical phenotyping data.

### BRIEF SUMMARY OF THE DISCLOSURE

There is disclosed a panel consisting of fourteen metabolites, which included six metabolites in the polyamine pathway, was identified that correctly discriminated lung cancer patients from controls with an area under the curve of 0.97 (95% CI: 0.875-1.0). The panel comprises the biomarkers valine, lysoPhosphatidylcholine acyl C18:2 (Lyso PC a18:2), decadienyl-L-carnitine (C10:2) phosphatidylcholine, acyl-alkyl C36:0 (PC aa C36:0), phosphatidylcholine diacyl C30:2 (PC aa C30:2), spermine, and diacetylspermine.

When used in conjunction with the SSAT-1/polyamine pathway, these metabolites may provide the specificity required for diagnosing lung cancer from other cancer types and could be used as a diagnostic and treatment monitoring tool.

### BRIEF DESCRIPTIONS OF THE DRAWINGS

FIG. 1 is a schematic showing the study design disclosed herein;

FIG. 2 is a hierarchical cluster analysis and heat map of correlation between the metabolites using training dataset (A); Partial Least-Squares Discriminant Analysis: training dataset (B); and test dataset (C);

FIG. 3 is a linear regression multivariate modeling with multiple combinations of metabolites 5 metabolites included valine, putrescine, PC.ae.C36.0, PC.aa.C32.2 and C10.2 (A) and 3 key metabolites Valine, Spermine and Ornithine (B);

FIG. 4 is a box plots under training data and validation datasets. X axis represents disease status; Y axis represents metabolite concentrations of corresponding metabolite. X axis represents disease status; Y axis represents metabolite concentrations of corresponding metabolite. Val: Valine; Met: Methionine Arg: Arginine, Org: Ornithine;

FIG. 5 is box plots showing the full range of Box plots of FIG. 4; and

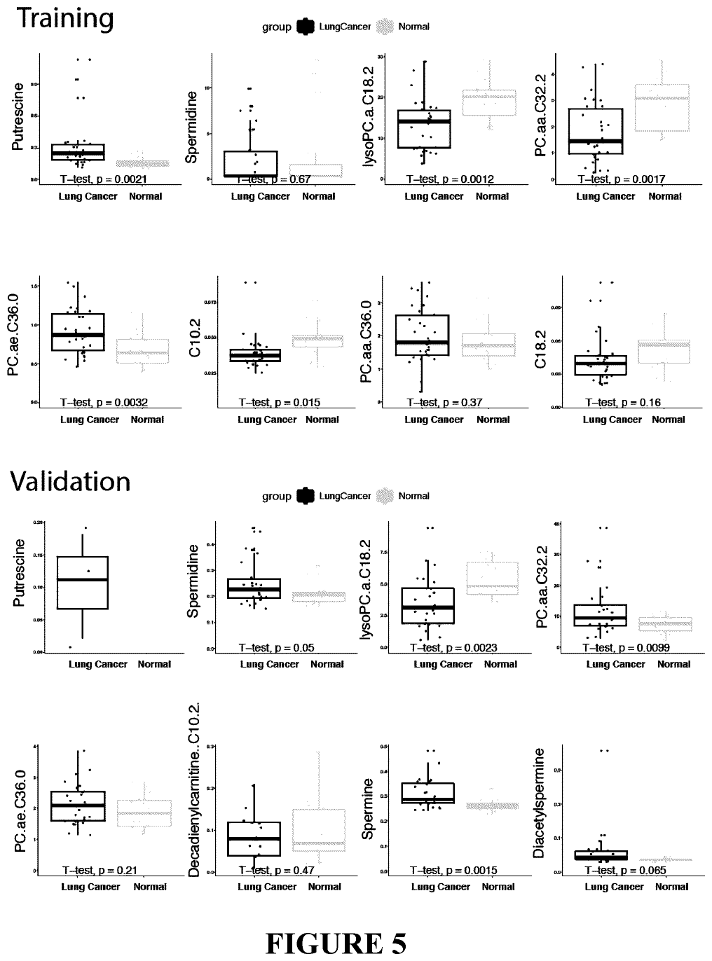

FIG. 6 is a statistical summary of samples and metabolite measures.

### DESCRIPTION OF THE SPECIFIC EMBODIMENTS

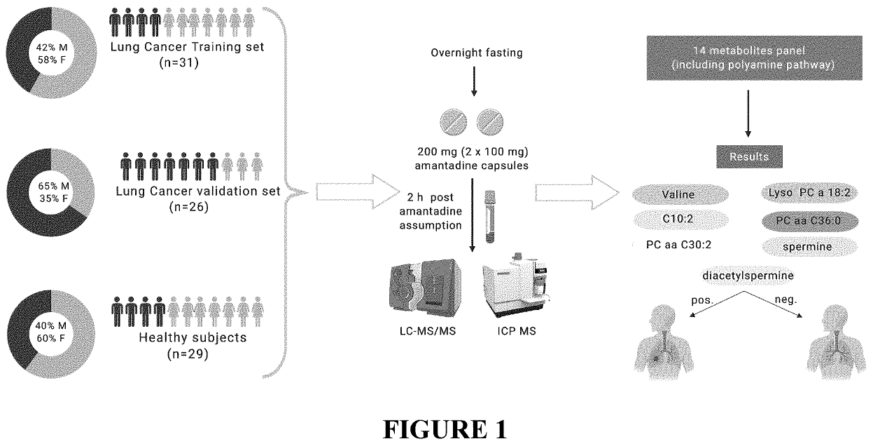

A study was undertaken to explore the use of amantadine as a means of measuring increased SSAT-1 activity. Patients with newly diagnosed and untreated cancer were recruited into the study. Lung cancer patients were recruited from the National Institute of Cancer Research & Hospital, Department of Medical Oncology, Mohakhali, Dhaka, Bangladesh. Healthy controls (n=29) were recruited from within same geographical location. The subjects used for this study were derived from a lung cancer patient cohort (n=80). All participants provided their approval with a signed informed consent for participation. Volunteers aged between 25 and 75 (median age: 52) years were included in the study as shown in FIG. 1. Exclusion criteria were declared as follows: alcohol consumption within 5 days of amantadine ingestion, previous adverse reaction to amantadine, currently pregnant or lactating, and liver or kidney disease. On the day of the study, blood samples were collected from overnight-fasted participants, prior to ingesting amantadine, and then requested to orally ingest 200 mg (2×100 mg) amantadine capsules (Mylan-Amantadine, amantadine hydrochloride, USP). Blood was collected 2 hours after amantadine administration. Following blood collection by venipuncture using sodium-oxalate coated vacutainer tubes, plasma was isolated by centrifugation (1000×g for 15 min) and plasma aliquoted (500 μL aliquots) and stored at −80 ° C. until analysis.

The study was approved by the Institutional Review Board of the Ministry of Health & Family Welfare, the People's Republic of Bangladesh (No. 115-15882). Clinical studies were completed under GCP and GLP conditions in accordance with local standards as well as the standards established by the Canadian Tri-Council Policies.

In the present study, plasma samples 2 hours post-ingestion of 200 mg of amantadine were analyzed. A high-throughput DI/LC-MS/MS based targeted quantitative assay for plasma samples has been developed and applied to measure a total of 15 metabolites (The Metabolomics Innovation Centre, Edmonton, AB, Canada), i.e., valine, putrescine, MTA (5′-Methylthioadenosine), Arginine, Ornithine, Spermidine, spermine, di-acetyl spermine, methionine, SAMe, N-acetyl Amantadine, Decadienylcarnitine (C10:2), PC aa C32:2, PC ae C36:0, lysoPC a C18:2 in plasma samples (Supplementary Materials, Table S3). The samples were analyzed using a kit-Optima™ LC/MS grade formic acid and HPLC grade water were purchased from Fisher Scientific (Ottawa, ON, Canada). L-valine, putrescine, arginine, ornithine, spermidine, spermine, methylthioadenosine, methionine, ammonium acetate, phenylisothiocyanate (PITC), HPLC grade pyridine, HPLC grade ethanol and HPLC grade acetonitrile (ACN) were purchased from Sigma-Aldrich (Oakville, ON, Canada). 1,2-dipalmitoleoyl-sn-glycero-3-phosphocholine, 1,2-distearoyl-sn-glycero-3-phosphocholine, 1-(9Z,12Z-octadecadienoyl)-sn-glycero-3-phosphocholine were purchased from Avanti Polar Lipids, Inc. (Alabaster, Ala., USA). N 1,N 12-diacetylspermine (hydrochloride) was purchased from Cayman Chemical (Ann Arbor, Mich., USA). Decadienylcarnitine was purchased from Medical Isotopes, Inc. (Pelham, N.H., USA). Tableisotope-labelled standards, including d 8-L-valine, 13 C 4-1,4-butanediamine, 13 C 6-arginine, and 5,5-d 2-L-ornithine were purchased from Cambridge Isotope Laboratories, Inc. (Tewksbury, Mass., USA). d 8 -spemidine, d 8 -spermine, and d 3-decanoyl-L-carnitine were purchased from IsoSciences (Ambler, Pa., USA). 15 N 5-adenosine was purchased from Medical Isotopes, Inc. (Pelham, N.H., USA). 1-linoleoyl-2-hydroxy-sn-glycero-3-phosphocholine-N,N,N-trimethyl-d 9 was purchased from Avanti Polar Lipids, Inc. (Alabaster, Ala., USA). Multiscreen “solvinert” filter plates (hydrophobic, PTFE, 0.45 μm, clear, non-sterile) and Nunc™ 96 Deep Well™ plates were purchased from Sigma-Aldrich (Oakville, ON, Canada) based assay (96-well plate format).

We have applied a targeted quantitative metabolomics approach to analyze the samples using a combination of direct injection (DI) mass spectrometry with a reverse-phase LC-MS/MS Kit. This kit in combination with an ABI 4000 Q-Trap (Applied Biosystems/MDS Sciex) mass spectrometer were used for the targeted identification and quantification of metabolites. The method used combines the derivatization and extraction of analytes, and the selective mass-spectrometric detection using multiple reaction monitoring (MRM) pairs. Isotope-labeled internal standards and other internal standards are used for metabolite quantification. The kit contains a 96 deep-well plate with a filter plate attached with sealing tape, and reagents and solvents used to prepare the plate assay. First 14 wells in the Kit were used for one blank, three zero samples, seven standards and three quality control samples provided with each Kit. Briefly, samples were thawed on ice and were vortexed and centrifuged at 13,000×g. 10 μL of each sample was loaded onto the center of the filter on the upper 96-well kit plate and dried in a stream of nitrogen. Subsequently, phenyl-isothiocyanate was added for derivatization. After incubation, the filter spots were dried again using an evaporator.

Extraction of the metabolites was then achieved by adding 300 μL of extraction solvent. The extracts were obtained by centrifugation into the lower 96-deep well plate, followed by a dilution step with kit MS running solvent. Mass spectrometric analysis was performed on an API4000 Qtrap® tandem mass spectrometry instrument (Applied Biosystems/MDS Analytical Technologies, Foster City, Canada) equipped with a solvent delivery system. The samples were delivered to the mass spectrometer by a LC method followed by a direct injection (DI) method. Data analysis was done using Analyst 1.6.2.

Raw metabolomics data were pre-processed, (1) the metabolites with more than 20% of missing values in all the groups were removed; (2) when missing values were less than 20%, they were imputed by half of minimum value for that specific metabolite. Two metabolites, methylthioadenosine (MTA) and S-adenosyl-L-methionine (SAMe), were removed from the analysis since they did not meet the quality control test. T-Test has been applied to examine the metabolites varying among normal and cancerous patients and false discovery rate (fdr) used for dealing with multiple testing error. Pearson's correlation coefficient (r) was used to investigate and measure the strength between the different metabolites in the training and validation datasets. The prediction ability of lung cancer was measured by each metabolite independently (univariate approach) and in combination (multivariate approach) using generalized linear regression model. Performance of the model was measured by area under the curve (AUC, ROC). The robustness of outcome is evaluated using 10-fold cross validation. In order to examine if further improvements could be achieved by combining more variable(s) to the primary model (with just top variable), the following was undertaken: (1) All the variables were ranked according to their AUC value (high to low). (2) One by one, each variable added to the high ranked variable and improvement of AUC was monitored. Different permutations and combinations were applied to find the best predictor with highest prediction ability. The entire analysis was performed using R/Bio-conductor. (https://www.r-project.org/).

The participant characteristics that include details about number of samples and clinical factors such as age and cancer subtypes are provided below in Tables A1, A2, and A3.

Table 1A is for first lung cancer cohort group A (training dataset), while Table 1B is the second cohort group B (validation dataset) which included mostly advanced stage (3+) lung cancers. Both cohorts A and B were from the n=80 lung cancer samples. The present analysis was based on 57 of 80 lung cancer patients. For the baseline, a cohort of healthy volunteers was chosen from recruited patients of n=29. A summary of both cancer and healthy cohorts is detailed in Table 1C below.

TABLE 1A
Training cohort

| Lung cancer subtypes | | | | | | |
|---|---|---|---|---|---|---|
| | Adeno- | Squamous | Non | Small | Metastasis | Age (yr) |
| | carcinoma (n) | (n) | Squamous (n) | cell (n) | (n) | Mean ± SD |
| Female (n = 18) | 13 | 5 | — | — | — | 47 ± 2 |
| Male (n = 13) | 9 | 3 | — | 1 | — | 56 ± 2 |
| Total (n = 31) | 22 | 8 | — | 1 | — | 52 ± 2 |

** Majority of patient at advanced stage 3+

TABLE 1B
Validation cohort

| Lung cancer subtypes | | | | | | |
|---|---|---|---|---|---|---|
| | Adeno- | Squamous | Non | Small | Metastasis | Age (yr) |
| | carcinoma (n) | (n) | Squamous (n) | cell (n) | (n) | Mean ± SD |
| Female (n = 9) | 2 | 2 | 1 | 2 | 2 | 54 ± 3 |
| Male (n = 17) | 4 | 5 | 5 | 3 | — | 56 ± 3 |
| Total (n = 26) | 6 | 7 | 6 | 5 | 2 | 56 ± 2 |

** Majority of patient at advanced stage 3+

TABLE 1C
Summary table of normal vs Lung cancer

| | Lung | Tranining | | Lung | Validation | |
|---|---|---|---|---|---|---|
| | Cancer (n) | Normal (n) | Pvalue | Cancer (n) | Normal (n) | Pvalue |
| Samples | 31 | 15 | | 26 | 15 | |
| Age: | Mean ± SD | Mean ± SD | | Mean ± SD | Mean ± SD | |
| Mean (Range) (yr) | 50.7 (27-75) | 43.7 (27-56) | 0.06 | 55.7 (27-70) | 43.7 (27-56) | 0.000213 |
| Sex: | | | | | | |
| Male | 13 | 6 | 0.75 | 17 | 6 | 0.12 |
| Female | 18 | 9 | | 9 | 9 | |

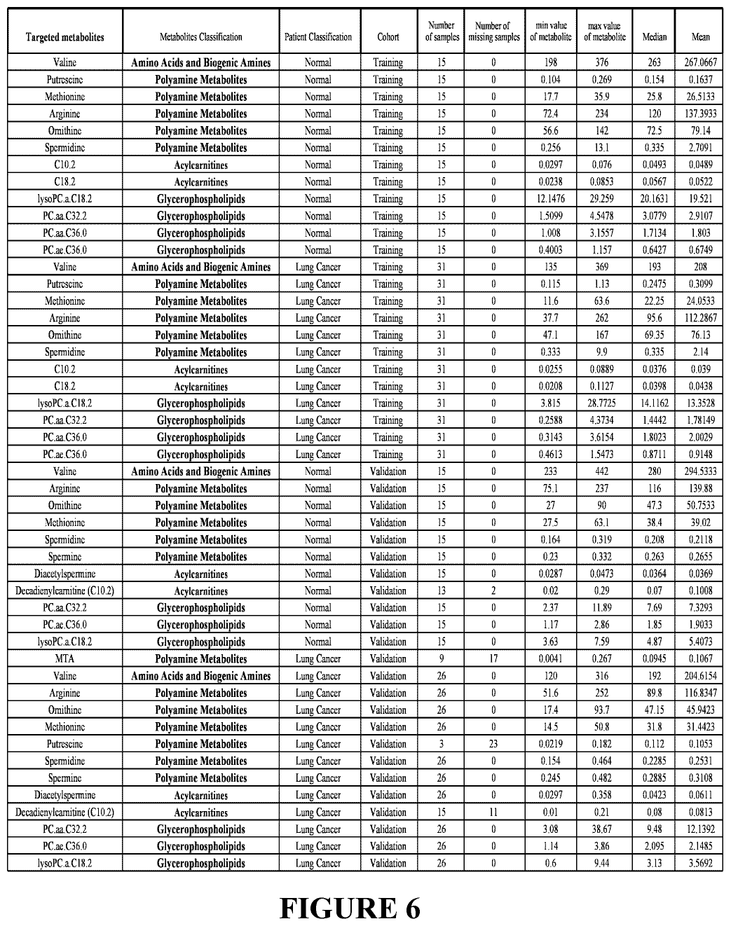

A statistical summary of targeted and measured metabolites along with number of samples in both training and validation cohort datasets is provided in Table 2 below. Targeted metabolites consist of polyamine and other endogenous metabolites comprised of amino acids, biogenic amines, acylcarnitines and glycerophospholipids as labelled in FIG. 6.

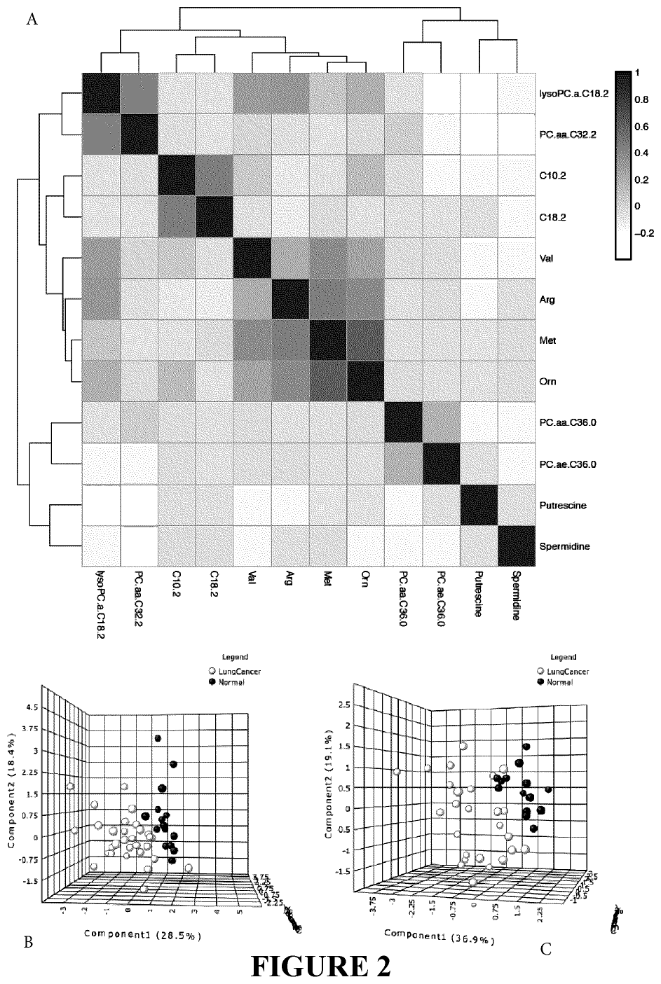

Hierarchical cluster analysis and heatmap of metabolite-metabolite correlation matrix was conducted and are shown in FIG. 2 on training dataset. A popular machine learning tool Partial Least-Squares Discriminant Analysis (PLS-DA) was used as a classifier to measure the differentiation between normal and lung cancer patients. PLS-DA is a chemometrics technique that is used to optimize separation between different groups of samples. There is evidence of good separation as depicted in FIG. 2 for both the training and validation cohort vs. the healthy group.

Each metabolite was tested independently to establish the prediction ability of outcome (lung cancer in this case) and then evaluated using training and validation datasets, respectively. Univariate summary between the datasets is presented in Table 3A below.

TABLE 3A
Univariate Summary Measure of each Metabolite

| | Training | | | Validation | | |
|---|---|---|---|---|---|---|
| | AUC | P value | FC | AUC | P value | FC |
| Arginine | 6.61E−01 | 1.30E−01 | −3.25E−01 | 6.64E−01 | 1.33E−01 | −3.26E−01 |
| C10.2 | 7.71E−01 | 9.44E−03 | −1.28E−01 | NA | NA | NA |
| C18.2 | 6.73E−01 | 1.85E−01 | −1.11E−01 | NA | NA | NA |
| Diacetylspermine | NA | NA | NA | 7.60E−01 | 2.59E−02 | 2.12E−01 |
| lysoPC.a.C18.2 | 7.73E−01 | 1.86E−03 | −6.60E−01 | 7.65E−01 | 3.16E−03 | −8.15E−01 |
| Methionine | 6.67E−01 | 4.01E−01 | −2.19E−01 | 6.85E−01 | 2.08E−02 | −3.44E−01 |
| Ornithine | 5.46E−01 | 6.97E−01 | −6.91E−02 | 5.69E−01 | 3.21E−01 | −1.70E−01 |
| PC.aa.C32.2 | 7.96E−01 | 2.12E−03 | −9.63E−01 | 6.94E−01 | 2.75E−02 | 5.91E−01 |
| PC.aa.C36.0 | 5.71E−01 | 4.13E−01 | 7.44E−02 | NA | NA | NA |
| PC.ac.C36.0 | 7.51E−01 | 6.69E−03 | 4.28E−01 | 6.12E−01 | 2.53E−01 | 1.64E−01 |
| Putrescine | 8.33E−01 | 2.03E−02 | 6.64E−01 | NA | NA | NA |
| Spermidine | 5.89E−01 | 6.12E−01 | 2.46E−01 | 6.29E−01 | 1.11E−01 | 1.90E−01 |
| Spermine | NA | NA | NA | 7.67E−01 | 5.20E−03 | 1.95E−01 |
| Valine | 8.04E−01 | 2.19E−03 | −3.83E−01 | 9.09E−01 | 2.58E−06 | −5.33E−01 |

AUC—Area Under the Curve;
FC—Fold Change,
NA—Not available

Valine and lysoPhosphatidylcholine acyl C18:2 (lyso PC a C18.2) were the most significant metabolites with false discovery rate (fdr)<0.01 for both the training and validation datasets when t-test analysis were performed. However, Decadienyl-L-carnitine (C10:2), Phosphatidylcholine diacyl C 32:2 (PC aa C32:2), Phosphatidylcholine diacyl C 36:0 (PC aa C36:0) and putrescine were significant in training data. Spermine and Diacetylspermine, which were detected only in validation data and showed significance at fdr<0.05. A summary of the t tests for both the training and validation datasets is shown in Table 3B below.

TABLE 3B
t-tests for key Metabolites using training
data (A) and validation data (B)

| Metabolites | t-statistic | P value | −log10 (p) | FDR |
|---|---|---|---|---|
| Training | | | | |
| Data A | | | | |
| Putrescine | 3.54E+00 | 9.88E−04 | 3.01E+00 | 6.44E−03 |
| Valine | −3.51E+00 | 1.07E−03 | 2.97E+00 | 6.44E−03 |
| lysoPC.a.C18.2 | 3.30E+00 | 1.97E−03 | 2.70E+00 | 7.89E−03 |
| PC.aa.C32.2 | −3.18E+00 | 2.72E−03 | 2.57E+00 | 8.15E−03 |
| PC.ac.C36.0 | 3.02E+00 | 4.27E−03 | 2.37E+00 | 9.32E−03 |
| C10.2 | −2.99E+00 | 4.66E−03 | 2.33E+00 | 9.32E−03 |
| Validation | | | | |
| Data B | | | | |
| Valine | −5.50E+00 | 2.58E−06 | 5.59E+00 | 2.58E−05 |
| lysoPC.a.C18.2 | −3.15E+00 | 3.16E−03 | 2.50E+00 | 1.58E−02 |
| Spermine | 2.96E+00 | 5.20E−03 | 2.28E+00 | 1.73E−02 |
| Methionine | −2.41E+00 | 2.08E−02 | 1.68E+00 | 4.59E−02 |
| Diacetyl spermine | 2.32E+00 | 2.59E−02 | 1.59E+00 | 4.59E−02 |
| PC aa. C32:2 | 2.29E+00 | 2.75E−02 | 1.56E+00 | 4.59E−02 |

−log 10 (p), FDR False Discovery Rate; t-statistic,

We tested multiple combinations of metabolites using linear regression multivariate modeling techniques to find the best predictor of lung cancer. Summary of the key metabolites is shown in Table 3C (training) and Table 3D (validation) below.

TABLE 3C
Training data, generalized linear regression multivariate
model statistics - key metabolites

| | Estimate | S.E. | P value |
|---|---|---|---|
| (Intercept) | 0.9438 | 0.2969 | 0.0029 |
| Valine | 0.0012 | 0.0011 | 0.2724 |
| Putrescine | −0.6203 | 0.2991 | 0.0447 |
| PC.aa.C32.2 | 0.1294 | 0.0563 | 0.027 |
| PC.aa.C36.0 | −0.2273 | 0.08 | 0.0071 |
| C10.2 | 10.3848 | 5.2603 | 0.0555 |

TABLE 3D
Validation data, generalized linear regression
multivariate model statistics - key metabolites

| | Estimate | S.E. | P Value |
|---|---|---|---|
| (Intercept) | 1.3407 | 0.4139 | 0.0025 |
| Valine | 0.0051 | 0.0009 | 1.84E−06 |
| Spermine | −2.8954 | 1.0274 | 0.0077 |
| Ornithine | −0.007 | 0.0036 | 0.0633 |

S.E., Standard Error

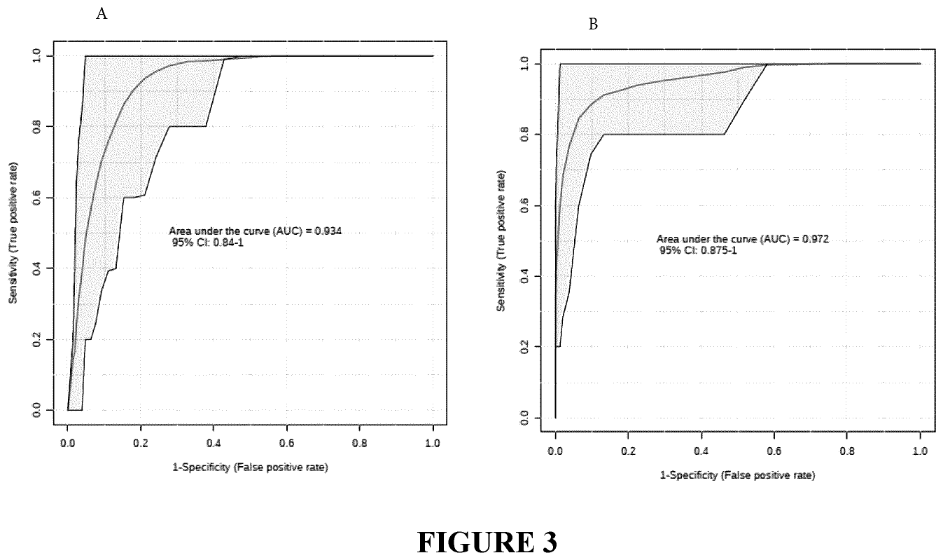

Using training data maximum AUC ROC achieved was 0.93 (0.84-1.0) with 5 metabolites which included valine, putrescine, PC.ae.C36.0, PC.aa.C32.2 and C10.2, as shown in FIG. 3A. In the validation data, maximum AUCROC achieved was 0.97 with three key metabolites valine, spermine and ornithine as shown in FIG. 3B. Based on these results, we found valine as the common predictor of lung cancer in both univariate and multivariate setting.

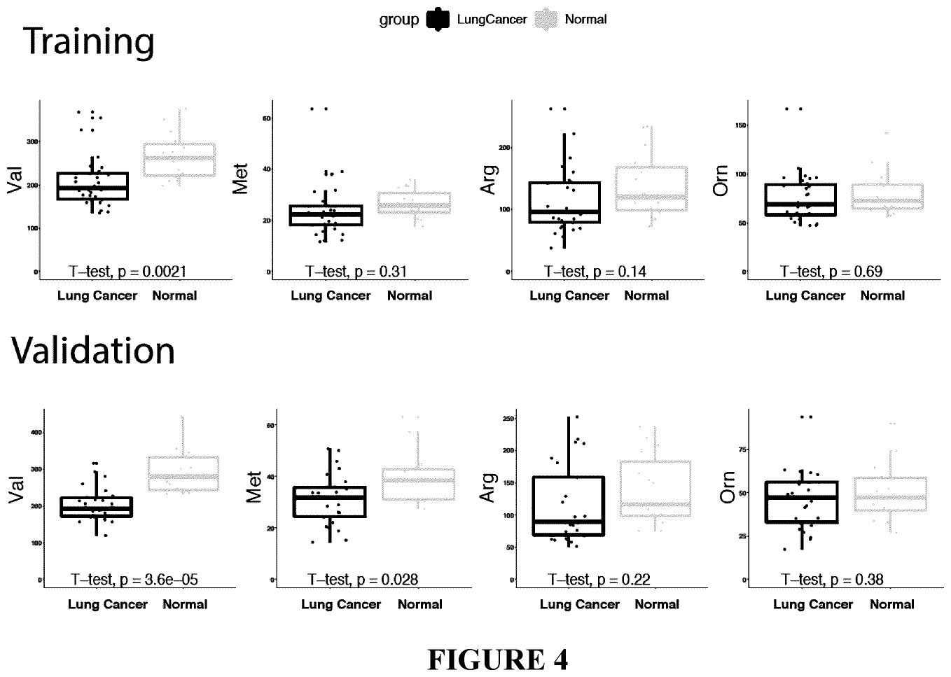

Box plots were constructed as a standardized approach to display the distribution concentrations of the different metabolites between lung cancer and healthy cohorts. In FIG. 4, the Box Plots demonstrates the different metabolite concentration distributions for both training and validation data for a few selected metabolites.

The purpose of this study was to evaluate whether combining a plasma panel of metabolites using a developed customized assay can further enhance and complement the SSAT-1 amantadine assay performance for detection of lung cancers. Patients that have tested with high levels of acetylamantadine can be further tested using this panel to better specify or rule in the type of cancer.

We utilized our finding to (1) identify a set of metabolites using a customized assay that may discriminate lung cancer from control following post-ingestion of amantadine (T2) and (2) understand the impact the new metabolites could have on improving the specificity of SSAT-1 amantadine assay. Analysis of the nature of the metabolites used for the discrimination of lung cancer from controls, as detailed earlier, reveals a panel of metabolites including valine, lysoPhosphatidylcholine acyl C18:2 (Lyso PC a18:2), decadienyl-L-carnitine (C10:2) phosphatidylcholine, acyl-alkyl C36:0 (PC aa C36:0), phosphatidylcholine diacyl C30:2 (PC aa C30:2), spermine, and diacetylspermine that can serve to discriminate between them. Changes in the concentrations of these metabolites are not surprising because lysophosphatidylcholines are membrane lipids known to be upregulated in lung cancer patients. In addition, higher concentrations of amino acids are detected during lung tumor development. For example, the high level of valine, leucine, and isoleucine found in lung tumors are required for energy production through the Krebs cycle. The metabolite diacetylspermine used in our study (even though only detected in the validation cohort) to discriminate patients from controls was found in plasma of patients as an excellent predictor of non-small cell lung cancer.

SSAT1, a key enzyme in the polyamine pathway, has been shown to be upregulated in different types of cancers. In recently published studies, acetyltransferase activity of the enzyme demonstrated that it may be useful as a diagnostic test for lung cancer by monitoring the conversion of the drug amantadine to its acetylated form.

The present disclosure provides a customized assay, which was comprised of some metabolites corresponding to the polyamine pathway and other metabolites, is highly applicable and feasible. The collective panel amplifies the signal and increases the tissue specificity of the SSAT-1 amantadine assay, which may serve as a promising lung cancer diagnostic tool.

It will be understood by a person skilled in the art that many of the details provided above are by way of example only and are not intended to limit the scope of the invention which is to be determined with reference to the following claims.

What is claimed is:

1. A biomarker panel for discriminating lung cancer wherein the biomarker panel comprises the biomarkers valine, lysoPhosphatidylcholine acyl C18:2 (Lyso PC a18:2), decadienyl-L-carnitine (C10:2) phosphatidylcholine, acyl-alkyl C36:0 (PC aa C36:0), phosphatidylcholine diacyl C30:2 (PC aa C30:2), spermine, and diacetylspermine.
